(19) United States
(12) Patent Application Publication
(10) Pub. No.: US 2006/0099251 A1
(43) Pub. Date: May 11, 2006

(54) Formulations of finasteride

(76) Inventor: Fjalar Johannsson, Reykjavik (IS)

Correspondence Address: WENDEROTH, LIND & PONACK, L.L.P., 2033 K STREET N. W., SUITE 800, WASHINGTON, DC, 20006-1021, US

(21) Appl. No.: 10/535,826

(22) PCT Filed: Nov. 21, 2003
(86) PCT No.: PCT/IS03/00034

§ 371 Date: Nov. 21, 2005

(30) Foreign Application Priority Data
Nov. 22, 2002 (IS) 6633

(51) Int. Cl. A61K 9/20

(52) U.S. Cl. 424/464

(57) ABSTRACT

This invention relates to pharmaceutical formulations of finasteride that include Gelucire®.

### FIELD OF THE INVENTION

The present invention relates to improved dissolution of finasteride tablet formulations.

### TECHNICAL BACKGROUND AND PRIOR ART

Finasteride, 17β-(N-tert-butyl carbamoyl)-4-aza-5α-androst-1 -en-3-one is a 5α-reductase inhibitor for use in treating acne, female hirsutism, male pattern hair loss (androgenic alopecia) and particularly benign prostatic hyperplasia. See for example U.S. Pat. No. 4,377,584 and Wilde and Goa, Finasteride, an update of its use in the management of symptomatic benign prostate hyperplasia, Drugs, Vol. 57, No. 4, April 1999, pp 557-581.

The preparation of useful formulations of finasteride is complicated since finasteride is practically insoluble in water. Hydrophobic therapeutic agents like finasteride present difficult problems in formulating such compounds for effective administration to patients. A well-designed formulation must, at a minimum, be capable of presenting a therapeutically effective amount of the hydrophobic compound to the desired absorption site, in an absorbable form.

WO 99/08666 and WO 99/08684 relate to novel solutions of aza steroid (finasteride included) in combination with fatty acid ester of glycerol or propylene glycol.

U.S. Pat. No. 6,294,192 relates to triglyceride-free pharmaceutical compositions for delivery of hydrophobic therapeutic agents.

In an attempt to improve the solubility of the active ingredient numerous wetting agents were tried. It has now been discovered that useful formulations of finasteride can be produced by use of Gelucire®.

Gelucire® is used in various applications including preparing sustained release pharmaceutical compositions, as described in the technical and patent literature. For example, U.S. Pat. No. 5,433,951 describes sustained release formulation containing captopril as the active ingredient.

U.S. Pat. No. 6,171,615 describes a stable sustained release theophylline formulation which is prepared by incorpurating theophylline into a semi-solid matrix comprising Gelucire.

U.S. Pat. No. 6,312,704 describes compositions providing enhanced bioavailability by mixing together a lipophilic phase, a surfactant, a cosurfactant an a pharmaceutical active ingredient. Gelucire 44/14 is one of the polyglycolized glycerides mentioned.

Sheen et al, Bioavailability of a Poorly Water-Soluble Drug from Tablet and Solid Dispersions in Humans, J. Pharm. Sci., Vol. 80, No. 7, July 1991, pp 712 to 714, discloses the use of Gelucire 44/14-polyethylene glycol (PEG) 400 mixture as carrier for REV 5901 (α-pentyl-3-(2-quinolinylmethoxy)benzenemethanol, a 5-lipoxygenase inhibitor) in a tablet. REV 5901 is a water-insoluble drug and the Gelucire 44/14-PEG 400 mixture provided improved dissolution thereof to effect faster release.

A. Ainaoiu, E. M. Ouriemchi, et al, Process of Drug Release with Oral Dosage Forms with a Lipidic Gelucire Matrix, Journal of Polymer Engineering, Vol. 17, No. 3, 1997, pp 245 to 255, discloses a study of drug release out of dosage forms made of the drug dispersed in Gelucire. The study concerns controlled release of the drug sodium salicylate.

D. Bidah, E. M Ouriemchi, et al, Diffusion Process of Drug Delivery from a Dosage Form wiht a Gelucire Matrix, No. 80, 1992, pp 145-149, describes a study of dosage forms having the property of delivering the drug at a controlled rate, with the drug, sodium salicylate, being dispersed in Gelucire, playing the role of a matrix, in syntetic gastric liquid.

None of the above references mention the use of Gelucire with finasteride.

### DETAILED DESCRIPTION

The invention provides a pharmaceutical formulation comprising finasteride, 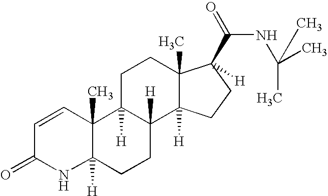
a wetting agent and the binding agent microcrystalline cellulose.

The pharmaceutical formulation of the present invention comprises 0.1-10 wt % of finasteride, 0-10 wt % of the wetting agent and 0-90 wt % of microcristalline cellulose.

The wetting agent may suitably be selected from Gelucire®, docusate sodium, sodium lauryl sulfate and polysorbate. Gelucire®is particularly preferred.

Gelucire® is a well-defined mixture of mono-, di- and triglycerides and mono- and di-fatty acid esters of polyethylene glycol, wherein the predominant fatty acid is lauric acid.

The binding agent may suitably also be selected from gelatin, dextrin, povidone or starch but microcrystalline cellulose is particularly preferred.

The formulation may additionally comprise a further pharmaceutically active compound, such as epristeride and zanosterone; filler material such as cellulose and starch; and lubricant such as magnesium stearate.

These formulations may also comprise a finasteride analogs instead of finasteride.

### EXAMPLES

### Example 1

The following materials were combined by wet granulation to produce 5 mg finasteride tablets:

| | |
|---|---|
| Finasteride | 5.0 mg |
| Lactose monohydrate powder | 103.3 mg |
| Cellulose microcrystalline | 15.0 mg |
| Starch maiza pregel. | 15.0 mg |
| Gelucire 44/14 ® | 3.0 mg |
| Sodium starch glyc. | 7.5 mg |
| Talcum | 0.4 mg |
| Magnesium stearate | 0.8 mg |

### Example 2

The following materials were combined by wet granulation to produce 5 mg finasteride tablets:

| | |
|---|---|
| Finasteride | 5.0 mg |
| Lactose monohydrate powder | 88.7 mg |
| Cellulose microcrystalline, | 30.0 mg |
| Starch maiza pregel. | 15.0 mg |
| Gelucire 44/14 ® | 3.0 mg |
| Sodium starch glyc. | 7.5 mg |
| Magnesium stearate | 0.8 mg |

### Example 3

The following materials were combined by wet granulation to produce 5 mg finasteride tablets:

| | |
|---|---|
| Finasteride | 5.0 mg |
| Lactose monohydrate powder | 104.4 mg |
| Cellulose microcrystalline | 15.0 mg |
| Starch maiza pregel. | 15.0 mg |
| Gelucire 44/14 ® | 2.3 mg |
| Sodium starch glyc. | 7.5 mg |
| Magnesium stearate | 0.8 mg |

### Example 4

The following materials were combined by wet granulation to produce 5 mg finasteride tablets:

| | |
|---|---|
| Finasteride | 5.0 mg |
| Lactose monohydrate powder | 79.7 mg |
| Cellulose microcrystalline | 37.5 mg |
| Starch maiza pregel. | 15.0 mg |
| Gelucire 44/14 ® | 4.5 mg |
| Sodium starch glyc. | 7.5 mg |
| Magnesium stearate | 0.8 mg |

### Example 5

The following materials were combined by wet granulation to produce 5 mg finasteride tablets:

| | |
|---|---|
| Finasteride | 5.0 mg |
| Lactose monohydrate powder | 105.6 mg |
| Cellulose microcrystalline, | 15.0 mg |
| Starch maiza pregel. | 15.0 mg |
| Gelucire 44/14 ® | 1.1 mg |
| Sodium starch glyc. | 7.5 mg |
| Magnesium stearate | 0.8 mg |

### Example 6

The following materials were combined by wet granulation to produce 5 mg finasteride tablets:

| | |
|---|---|
| Finasteride | 5.0 mg |
| Lactose monohydrate powder | 105.2 mg |
| Cellulose microcrystalline | 15.0 mg |
| Starch maiza pregel., | 15.0 mg |
| Gelucire 44/14 ® | 1.5 mg |
| Sodium starch glyc. | 7.5 mg |
| Magnesium stearate | 0.8 mg |

### Example 7

The following materials were combined by wet granulation to produce 5 mg finasteride tablets:

| | |
|---|---|
| Finasteride | 5.0 mg |
| Lactose monohydrate powder | 106.3 mg |
| Cellulose microcrystalline | 15.0 mg |
| Starch maiza pregel. | 15.0 mg |
| Gelucire 44/14 ® | 0.4 mg |
| Sodium starch glyc. | 7.5 mg |
| Magnesium stearate | 0.8 mg |

### Example 8

Disintegration time and friability of finasteride formulations

| | Disentegrationa time | Friability |
|---|---|---|
| | [min:sec] | [%] |
| Example 1 | 1:40 | 0.93 |
| Example 2 | 3:00 | 0.30 |
| Example 3 | 2:30 | 0.60 |
| Example 4 | 2:40 | 0.46 |
| Example 5 | 1:45 | 0.66 |
| Example 6 | 1:00 | 0.40 |
| Example 7 | 1:00 | 0.33 |

### Example 9

Comparision of dissolution speed between formulations including the wetting agents Gelucire®, polysorbate 80, sodium lauryl sulfate and docusate sodium.

| | FI111-15 | FI111-4 | FI111-7 | FI111-8 |
|---|---|---|---|---|
| Finasteride | 5.0 | 5.0 | 5.0 | 5.0 |
| Lactose monohydrate powder | 105.2 | 106.1 | 103.3 | 105.9 |
| Cellulose microcrystalline | 15.0 | 15.0 | 15.0 | 15.0 |
| Starch maiza pregel. | 15.0 | 15.0 | 15.0 | 15.0 |
| Gelucire 44/14 ® | 1.5 | | | |
| Polysorbate 80 | | 0.4 | | |
| Sodium lauryl sulfate | | | 3.0 | |
| Docusate sodium | | | | 0.4 |
| Sodium starch glyc. | 7.5 | 7.5 | 7.5 | 7.5 |
| Talcum | | | 0.4 | 0.4 |
| Magnesium stearate | 0.8 | 0.8 | 0.8 | 0.8 |

TABLE 1
% dissolved finasteride with different
wetting agents

| | | | FI111-7 | FI111-8 |
|---|---|---|---|---|
| | FI111-15 | FI111-4 | Sodium | Docusate |
| time [min] | Gelucire | Polysorbate | lauryl sulfate | sodium |
| 0.0 | 0.0 | 0.0 | 0.0 | 0.0 |
| 5.0 | 29.5 | 28.3 | 45.8 | 68.2 |
| 7.0 | 43.2 | 45.3 | 62.4 | 80.3 |
| 10.0 | 50.3 | 54.7 | 71.8 | 85.8 |
| 15.0 | 56.9 | 63.6 | 79.5 | 89.1 |
| 20.0 | 61.0 | 69.8 | 83.4 | 92.1 |
| 30.0 | 67.5 | 74.8 | 87.2 | 92.1 |
| 45.0 | 73.4 | 80.4 | 89.5 | 93.1 |
| 60.0 | 76.0 | 83.7 | 90.9 | 92.6 |

[image]

Examples 2-7 show various concentrations of Gelucire® and microcrystalline cellulose.

Example 8 shows results in disintegration time and friability for the formulations in Examples 2-7.

Example 9 shows different dissolution profiles for different wetting agents in the formulation.

By using Gelucire 44/14® the dissolution profile of the finasteride improved to a satisfactory level. However the binding of the tablets decreased as the quantity of Gelucire increased. In example 1 (2% Gelucire concentration) the friability of the tablets became almost 1%. In an achivement to improve the binding, the levels of microcrystalline cellulose were increased.

1. A pharmaceutical tablet formulation comprising:
a. of finasteride;
b. a wetting agent selected from the group consisting of Gelucire®, sodium lauryl sulfate and polysorbate,
c. a binding agent selected from the group consisting of gelatin, dextrin, povidone starch and microcrystalline cellulose,
optionally in combination with other excipients.

2. The tablet formulation of claim 1, wherein the wetting agent is Gelucire.

3. The tablet formulation of claim 2, wherein the Gelucire is Gelucire 44/14.

4. The tablet formulation of claim 1, which comprises finasteride, Gelucire®, microcrystalline cellulose, lactose monohydrate powder, starch, sodium starch glycolate and magnesium stearate.

5. The tablet formulation of claim 1 wherein the binding agent comprises microcrystalline cellulose.

6. The tablet fomulation of claim 1, wherein the wt/wt ratio of the wetting agent and the binding agent is in the range of 0.01-1.0.
